(19) United States
(12) Patent Application Publication
(10) Pub. No.: US 2025/0102503 A1
(43) Pub. Date: Mar. 27, 2025

(54) LATERAL FLOW IMMUNOASSAY DEVICE AND SYSTEM FOR ASSESSING ASSAY FLOWABILITY AND METHODS OF USING THE SAME

(71) Applicant: Quanovate Tech Inc., San Ramon, CA (US)

(72) Inventors: Sara Marska-Maj, San Ramon, CA (US); Sylvia M. Kang, Castro Valley, CA (US); Zheng Yang, Castro Valley, CA (US); Chang-Yi Huang, Calamvale (AU)

(21) Appl. No.: 18/892,000

(22) Filed: Sep. 20, 2024

Related U.S. Application Data
(60) Provisional application No. 63/584,459, filed on Sep. 21, 2023.

(51) Int. Cl. G01N 33/543; B01L 3/00; G01N 33/74

(52) U.S. Cl. CPC G01N 33/54388; B01L 3/5023; G01N 33/74; B01L 2300/0654; B01L 2300/069

(57) ABSTRACT

Systems and methods for detecting an analyte include test wands configured to employ immunochromatographic assay with new fluorescent labeling technology, a reading device that can specifically identify signals from wands and sync information to the mobile devices. The systems may quantitatively detect progesterone metabolites and hormones in urine to track the level changes, confirm ovulation, and/or evaluate menstruation status. The systems and methods assess and track an individual's ovulation cycle by monitoring a hormone pattern of that specific individual and establishing a dynamic threshold as a baseline for changes in the specific subject's hormones. The systems and methods further include enhancing the accuracy and reliability of lateral flow immunoassays through generation of signal curves plotting signal intensity versus time and comparative analysis of signal intensity over time to assess flowability within an assay and improve overall assay performance.

### FIELD OF THE INVENTION

The present invention relates to bioassays, specifically to lateral flow immunoassays, for detecting the level of presence or absence of analytes in a biological sample to assess a condition or status of a subject, and to systems and methods for assessing and controlling flowability of a biological sample along the lateral flow immunoassays.

### BACKGROUND OF THE INVENTION

Lateral flow assay devices are critical in point-of-care diagnostics due to their ability to provide rapid and cost-effective mechanisms for detecting the presence or absence of analytes in a sample as well as their straightforward operation.

Lateral flow assay devices have been used to assess the ovulation or menstrual cycle of a subject, conventionally, by detecting progesterone metabolites in urine by calculating a level of pregnanediol glucuronide (PdG) and evaluating the calculated level relative to a predetermined PDG threshold level. Changes in the calculated level of pregnanediol glucuronide may be observed, for example, based on differences in color or color intensity of a detectable label relative to a reference color, or based on a simple image, such as the display of one or more lines. Examples of such conventional methods are disclosed by Ecochard, R., et al. “Use of urinary pregnanediol 3-glucuronide to confirm ovulation.” Steroids 78.10 (2013): 1035-1040; and Koczula, K. M., and A. Gallotta. “Lateral flow assays.” Essays Biochem 60 (2016): 111-120.

More recently, Beckley (U.S. Pat. Nos. 11,029,321 and 11,061,026) has taught the use of a lateral flow assay comprising both a sandwich assay and a competitive assay to detect the presence or absence of at least PdG in a non-scrum bodily fluid. The lateral flow assay taught in this instance includes a first conjugate pad saturated with monoclonal anti-pregnanediol glucuronide (anti-PdG) antibodies conjugated to a first visual label in a concentration of 1-10 μg/mL; a second conjugate pad saturated with selected antibodies chosen from anti-luteinizing hormone, anti-follicle stimulating hormone, and anti-human chorionic gonadotropin, and conjugated to a second visual label; and a membrane comprising a first testing zone and a second testing zone. The membrane provides a first observable result for the absence or presence of PdG at or above a PdG threshold of 3-20 μg/ml, as indicated by a color display at the first visual label in the first testing zone and provides a second observable result for the absence or presence of the selected hormone(s), as indicated by a color display at the second visual label in the second testing zone.

Despite the advances provided to date in the art, there remain a number of shortcomings in the current state of the art. For example, conventional approaches provide only a means for analyzing a hormone metabolite at a single moment in time, without means for tracking a pattern of test results. The conventional systems and methods are also subject to analysis errors due to inappropriate selection of antibodies and type of carrier proteins, and the test results are subject to misinterpretation due to the use of results displays that may be easily misread by a user (e.g., a number of lines or variances in color). Conventional systems and methods are also subject to incorrect and/or inconsistent results due to failures or variations in fluid flowability of a biological sample along the lateral flow immunoassay devices.

Thus, there remains a need in the art for immunoassay devices that can provide superior test results to a user while also reducing the likelihood of erroneous results and misinterpretation by users.

### SUMMARY OF THE INVENTION

The present invention is inclusive of a system that includes test wands in the form of lateral flow immunoassay devices configured to employ immunochromatographic assay with new fluorescent labeling technology and a signal analyzer having a reading device that can identify the specific fluorescent signals from the test wands. The signal analyzer is adapted to determine test results from the detected fluorescent signals and to sync information of the test to application software and remote devices. Systems according to the present invention may be used, for example, to quantitatively detect progesterone metabolites in urine to track the level changes, confirm ovulation, and/or evaluate menstruation status. Systems according to the present invention may also use signal analyses methods to ensure proper fluid flow of a test sample through the test wand to thereby improve consistency and accuracy of test results.

A lateral flow immunoassay system comprises an immunoassay device and a signal analyzer with a computational module. The immunoassay device has a test strip for reception and migration of a test sample therealong, the test strip comprising: a sampling pad for reception of a test sample, conjugate pad carrying optical labels (e.g., fluorescent labels) conjugated to antibodies specific to one or more target analytes in the test sample, and one or more test zones for capture of one or more target analytes. The signal analyzer has a scanning mechanism (e.g., a fluorescence scanner) configured to perform multiple optical scans of the one or more test zones of the test strip to detect optical signals from optical labels on one or more captured target analytes. The computational module is configured to generate signal detection curves based on optical signals detected from optical scans of the one or more test zones, and to compare signal detection curves to assess flow characteristics of the test sample along the test strip and determine the signal intensities and concentrations of the target analyses.

The computational module may be configured to generate signal detection curves plotting optical signal intensity against positional units along the test strip length for each optical scan, with the computational module configured to compare signal detection curves to identify variations in flow dynamics and analyte distribution of the test sample along the immunoassay device and to adjust assay parameters in real-time based on the determined flow characteristics of the test sample. The computational module may be configured to adjust assay parameters that include the performance of additional optical scans of the one or more test zones and changing an interval of time between the additional optical scans. The computational module may be further configured to detect occurrence of errors in an immunoassay test based on the determined flow characteristics of the test sample.

A method of performing a lateral flow immunoassay comprises: introducing a test sample onto a sampling pad of a test strip; allowing the test sample to migrate along the test strip via capillary action, with the test sample migrating through a conjugate pad of the test strip for labelling of one or more target analytes with an optical label (e.g., a fluorescent label) and subsequently through one or more test zones of the test strip for capture of one or more target analytes; performing multiple optical scans (e.g., via a fluorescence scanner) of the one or more test zones to detect emission signals from optical labels attached to the one or more target analytes captured at the one or more test zones, with individual optical scans performed at different time periods; generating signal detection curves based on the emission signals detected from the optical labels in the multiple optical scans; and comparing signal detection curves to assess flow characteristics of the test sample along the test strip and determining the signal intensities and concentrations of the target analyses.

The method may further comprise the generation of signal detection curves that plot optical label intensity against positional units along the test strip length for each optical scan. The method may further comprise adjusting assay parameters based on the determined flow characteristics in real-time, with the adjusted parameters including performing additional optical scans of the one or more test zones and changing an interval of time between the additional optical scans. The method may further comprise detecting the occurrence of errors in an immunoassay test based on the determined flow characteristics of the test sample.

Both the foregoing general description and the following detailed description are exemplary and explanatory only and are intended to provide further explanation of the invention as claimed. The accompanying drawings are included to provide a further understanding of the invention; are incorporated in and constitute part of this specification; illustrate embodiments of the invention; and, together with the description, serve to explain the principles of the invention.

### BRIEF DESCRIPTION OF THE DRAWINGS

Further features and advantages of the invention can be ascertained from the following detailed description that is provided in connection with the drawings described below:

FIGS. 1A-1B show an immunoassay device according to the present invention, in which: FIG. 1A shows a top plan view of the immunoassay device; and FIG. 1B shows a bottom plan view of the immunoassay device;

FIGS. 2A-2B show a signal analyzer according to the present invention, in which: FIG. 2A shows a front perspective view of the signal analyzer; and FIG. 2B shows a rear elevation view of the signal analyzer;

FIGS. 3A-3D show examples of an interface to a signal processing program, in which: FIG. 3A shows a home page display; FIG. 3B shows a historical test results display; FIG. 3C shows a hormone data display; and FIG. 3D shows a hormone cycle editing display;

FIG. 4 shows a first exploded view of the immunoassay device in FIGS. 1A-1B;

FIG. 5 shows a second exploded view of the immunoassay device in FIG. 4;

FIG. 6 shows an exploded view of the signal analyzer in FIGS. 2A-2B;

FIG. 7 shows a first view of an immunoassay device inserted into a signal analyzer;

FIG. 8 shows a second view of an immunoassay device inserted into a signal analyzer;

FIG. 9 shows a third view of an immunoassay device inserted into a signal analyzer;

FIG. 10 shows an example of an optical-mechanical detector module of the signal analyzer in FIGS. 2A-2B; and

FIGS. 11A-11B show examples of fluorescence intensity curves according to the present invention, in which: FIG. 11A shows measurements of a single peak; and FIG. 11B shows assessment of relative positioning between multiple peaks.

FIGS. 12A-12E show examples of fluorescence intensity curves generated from signals measured from an immunoassay device with four test zones at different time intervals, in which: FIG. 12A shows a curve generated at a time interval T=0; FIG. 12B shows a curve generated at a time interval T=1 minute; FIG. 12C shows a curve generated at a time interval T=5 minutes; FIG. 12D shows a curve generated at a time interval T=10 minutes; and FIG. 12E shows a curve generated at a time interval T=15 minutes.

FIGS. 13A-13B show signal intensities measured from a single test zone of an immunoassay device at several time intervals, in which: FIG. 13A shows an example with measurements of the signal intensity at five time intervals over a fifteen minutes; and FIG. 13B shows an example with measurements of the signal intensity at twenty-four time intervals over twenty-four minutes.

### DETAILED DESCRIPTION OF THE INVENTION

The following disclosure discusses the present invention with reference to the examples shown in the accompanying drawings, though does not limit the invention to those examples.

The use of any and all examples, or exemplary language (e.g., “such as”) provided herein is intended merely to better illuminate the invention and does not pose a limitation on the scope of the invention unless otherwise claimed. No language in the specification should be construed as indicating any non-claimed element as essential or otherwise critical to the practice of the invention, unless otherwise made clear in context.

As used herein, the singular forms “a,” “an,” and “the” include plural referents unless the context clearly dictates otherwise. Unless indicated otherwise by context, the term “or” is to be understood as an inclusive “or.” Terms such as “first”, “second”, “third”, etc. when used to describe multiple devices or elements, are so used only to convey the relative actions, positioning and/or functions of the separate devices, and do not necessitate either a specific order for such devices or elements, or any specific quantity or ranking of such devices or elements.

The word “substantially” as used herein with respect to any property or circumstance refers to a degree of deviation that is sufficiently small so as to not appreciably detract from the identified property or circumstance. The exact degree of deviation allowable in a given circumstance will depend on the specific context, as would be understood by one having ordinary skill in the art.

Use of the terms “about” or “approximately” are intended to describe values above and/or below a stated value or range, as would be understood by one having ordinary skill in the art in the respective context. In some instances, this may encompass values in a range of approx. +/−10%; in other instances, there may be encompassed values in a range of approx. +/−5%; in yet other instances values in a range of approx. +/−2% may be encompassed; and in yet further instances, this may encompass values in a range of approx. +/−1%.

It will be understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof, unless indicated herein or otherwise clearly contradicted by context.

The terms “individual”, “host”, “subject”, and “patient”, as may be used interchangeably herein, refer to a mammal, including, but not limited to, primates, for example, human beings, as well as rodents, such as mice and rats, and other laboratory animals.

Recitations of value ranges herein, unless indicated otherwise, serve as shorthand for referring individually to each separate value falling within the respective ranges, including the endpoints of the range, each separate value within the range, and all intermediate ranges subsumed by the overall range, with each incorporated into the specification as if individually recited herein.

Unless indicated otherwise, or clearly contradicted by context, methods described herein can be performed with the individual steps executed in any suitable order, including: the precise order disclosed, without any intermediate steps or with one or more further steps interposed between the disclosed steps; with the disclosed steps performed in an order other than the exact order disclosed; with one or more steps performed simultaneously; and with one or more disclosed steps omitted.

The present invention is inclusive of an immunoassay device (e.g., a test wand), an immunoassay signal analyzer (e.g., a test reader) and signal processing program (e.g., application software). The immunoassay device is a lateral flow immunochromatographic assay with a nitrocellulose membrane having fluorescent labeling technology for the detection and labeling of one or more analytes with one or more fluorescent labels. The signal analyzer is configured to identify and read immunoassay signals (e.g., light signals emitted by the fluorescent dye materials of the fluorescent labels) from the immunoassay device, and to save and sync test results (e.g., concentration levels of one or more analytes being tested) with local and/or remote storage. Saving, syncing, and storage of test results is achieved through use of the signal processing program, which may include programming stored locally in the signal analyzer as well as programming stored remotely in one or more user devices/systems (e.g., mobile phone, tablet, personal computing device, etc.) and/or one or more remote service devices (e.g., service hosting servers; cloud servers; etc.).

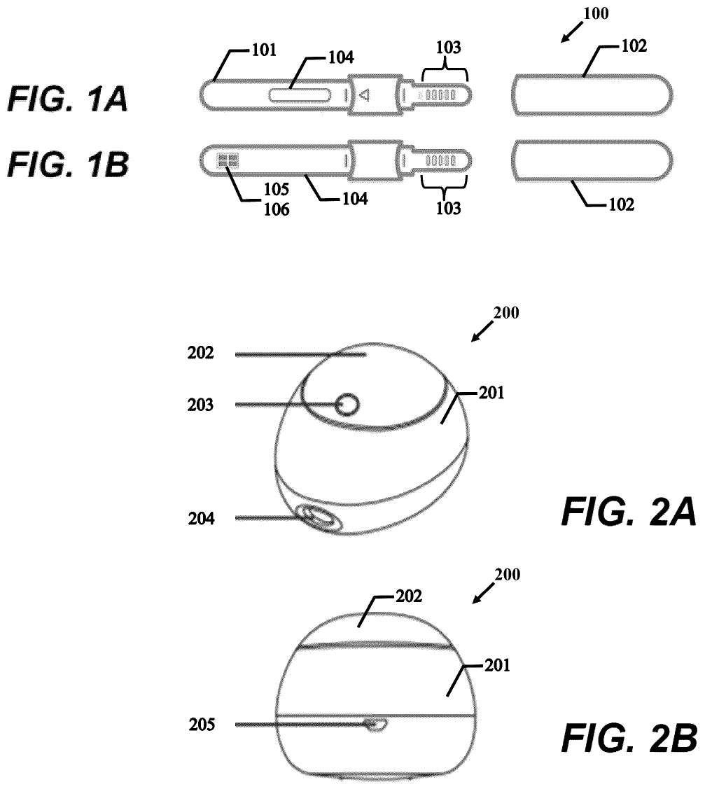

FIGS. 1A-1B show an example of an immunoassay device 100 according to the present invention, with a top plan view shown in FIG. 1A and a bottom plan view shown in FIG. 1B. The immunoassay device 100 includes a casing 101 enclosing an internal test strip from an external environment and a releasable cap 102. A sample collection region 103 is provided at a first end of casing 101, with one or more openings provided at the sample collection region 103 to enable delivery of a test sample to the internal test strip. At least one reading window 104 is provided at the top side of casing 101 to enable the transmission of test signals from the internal test strip. A chip cavity 105 containing a microchip 106 (e.g., calibration chip) is provided at the bottom side of casing 101. Cap 102 is adapted for selectively engaging casing 101 to cover and expose the sample collection region 103. The cap 102 may optionally be adapted for selective engagement to cither end of casing 101 (e.g., to cover the sample collection region 103 when engaged at a first end, or for convenient storage when engaged at a second end). The reading window 104 is adapted to align with test zones provided along the internal test strip to expose the test regions for transmission of test signals for reading by a signal analyzer. Casing 101 may optionally include a closure means (e.g., a sliding cover panel) for selectively covering and exposing the openings that enable transmission of the test signals. The microchip 106 stores information for use in immunoassay testing and is adapted for communicating with a calibration module in the signal analyzer for reading of the stored information.

FIGS. 2A-2B show an example of signal analyzer 200 according to the present invention, with a front perspective view shown in FIG. 2A and a rear elevation view shown in FIG. 2B. The signal analyzer 200 has a housing 201 with a display screen 202 at a top side thereof and a user interface 203 (e.g., a button) for a user to input data to a computing device within the signal analyzer 200. An insertion slot 204 (e.g., test wand slot) is provided at a front side of housing 201 for insertion of an immunoassay device 100, and a communications port 205 (e.g., USB port) is provided at a back side of housing 201.

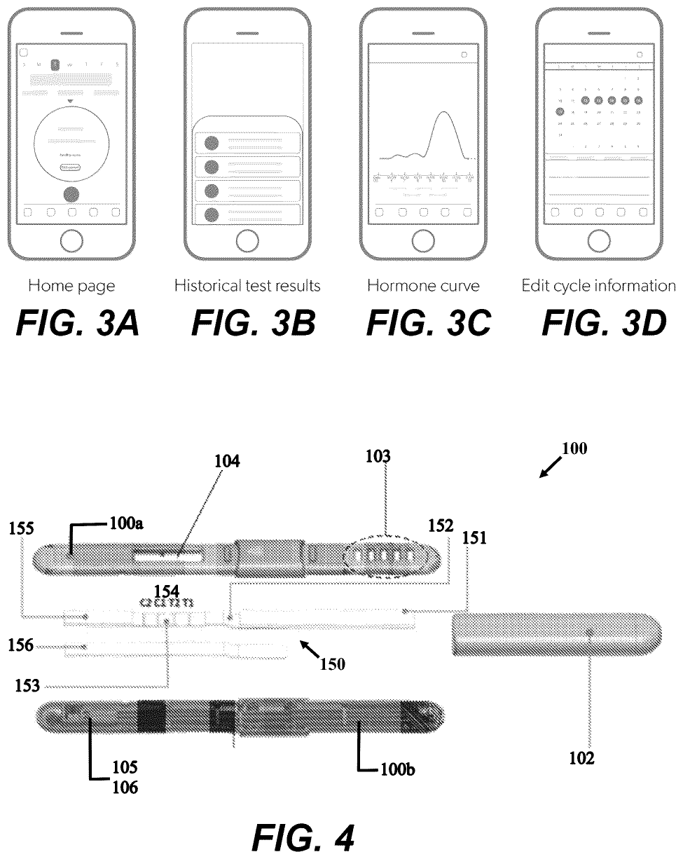

FIGS. 3A-3D show examples of a signal processing program interface, with a home page display shown in FIG. 3A, a historical test results display shown in FIG. 3B, a hormone data display shown in FIG. 3C, and a hormone cycle editing display shown in FIG. 3D.

In use, the immunoassay device 100 receives a test sample, labels one or more predetermined, targeted analyte(s) with one or more fluorescent labels, and captures the one or more labeled targeted analytes through immobilization at one or more testing zones. The signal analyzer 200 is then used to detect light signals from the fluorescent labels of the one or more targeted analytes captured in the immunoassay device 100 and determine a level of the targeted analyte(s) based on the detected fluorescent signals. In a preferred example, a system comprising an immunoassay device 100 and signal analyzer 200 according to the present invention is adapted for concurrently and quantitatively determining levels of luteinizing hormone, follicle-stimulating hormone, estradiol metabolites, and progesterone metabolites in urine to thereby track changes in hormone levels throughout a subject's menstrual cycle. Optionally, the system may be further adapted to track a fertility-related health status and trend, diagnose fertility-related conditions, track the progress of fertility-related treatments, and predict the outcome of fertility-related treatments.

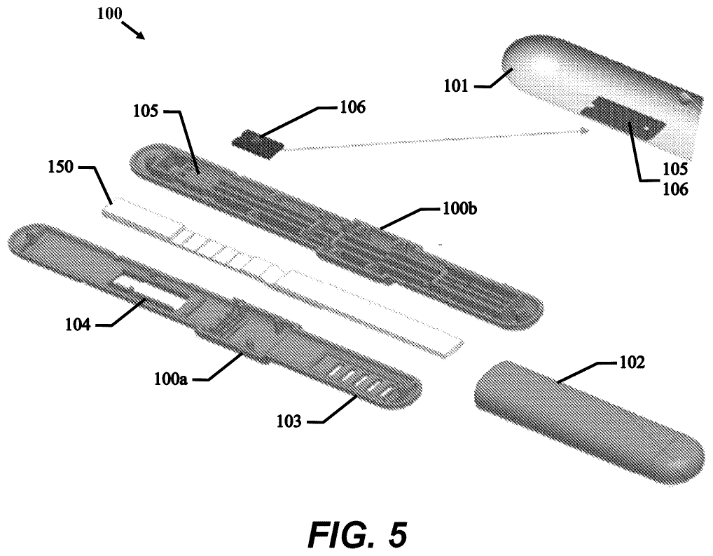

As shown in FIGS. 4-5, casing 101 of immunoassay device 100 includes a top panel 101a and a bottom panel 101b that define an internal space enclosing a test strip 150. The test strip 150 includes a sampling pad 151 for receiving a test sample (e.g., a bodily fluid sample), a conjugate pad 152 saturated with multiple preparatory chemical configurations and multiple antibodies that are conjugated with fluorescent dyes, a nitrocellulose membrane 153 comprising one or more testing zones 154, and an absorbent pad 155 (e.g., wicking pad) at an end opposite the sampling pad 151 to promote lateral flow of the test sample along the length of the test strip 150. Test strip 150 is positioned within casing 101 such that the sampling pad 151 aligns with openings at the sample collection region 103 and the test zones 154 align with the reading window 104. The test strip 150 further includes a stiffened backing support 156 to maintain a level orientation that promotes uniform flow of the test sample therealong. Optionally, test strip 150 may further include one or more control zones (not shown) positioned between the conjugate pad 152 and the absorbent pad 155, with multiple potential arrangements of the control zone(s) relative to the testing zones 154.

In a preferred example, immunoassay device 100 is configured for use with test samples in the form of biological samples, such as urine, blood, saliva, and other such bodily fluids. Conjugate pad 152 is saturated, but not limited to, with one or more antibodies chosen from anti-luteinizing hormone (anti-LH), anti-estrone-3-glucuronide (anti-E3G), anti-pregnanediol 3-glucuronide (anti-PdG) and anti-follicle-stimulating hormone (anti-FSH) antibodies. The antibodies at conjugate pad 152 are each conjugated in advance with one or more fluorescent dyes, which may include a single type of fluorescent dye or multiple different types of fluorescent dyes.

A separate testing zone 154 is provided for each antibody saturated on the conjugate pad 152, with respective testing zones 154 at three different locations on the membrane 153 for each of the anti-LH antibodies, anti-E3G antibodies, and anti-PdG antibodies in the illustrated example. Each testing zone 154 is loaded with capture reagents for capturing cither non-conjugated or conjugated complexes of the anti-LH antibodies, anti-E3G antibodies, and anti-PdG antibodies respectively. For example, there may be provided three separate test zones 154a, 154b, and 154c, provided respectively with: anti-LH antibodies, E3G-protein conjugated complexes, and PdG-protein conjugated complexes in the illustrated example.

The fluorescent dyes have specific excitation and emission spectra such that, when irradiated with light of a first specific wavelength W1 (excitation light), the fluorescent material emits light (emission light) with a second specific wavelength W2 different from that of the first specific wavelength W1. The wavelength W2 of the emission light is not visible to the unaided human eye. The intensity of the emission light from the fluorescent dyes is proportional to the concentration of the fluorescent dyes, and the concentration of the fluorescent dyes is proportional to the antibodies to which the fluorescent dyes are conjugated. The concentration of the antibodies to which the fluorescent dyes are conjugated are proportional, directly or inversely, to the analytes targeted for detection within the test sample. With these relationships, the intensity of the emission light from the fluorescent dyes is proportional, directly or inversely, to the targeted analytes, thereby enabling estimation of a concentration of the targeted analytes in a test sample via measurement of an intensity of emission light from the fluorescent labels. In this way, a system comprising the immunoassay device 100 in combination with the signal analyzer 200 enables quantitative testing for targeted analytes.

The microchip 106 (e.g., calibration chip, or any other storage-communication device) embedded in the casing 101 may contain information about the immunoassay device 100, which may include one or more of: the device type (e.g., model type, use type, etc.) of the immunoassay device 100; the type of test sample(s) for which the immunoassay device 100 is intended for use with; the analytes targeted for identification in the test sample(s); the testing intended for use in identifying the targeted analytes; predetermined parameters for calculating a concentration for each targeted analyte (e.g., standard curve data); device lifetime dates (e.g., date of manufacture, expiration date, etc.). The microchip 106 is adapted for reading by a calibration module of the computing device at the signal analyzer 200 when the immunoassay device 100 is inserted into the insertion slot 204 of the signal analyzer 200. The signal analyzer 200 uses the information stored in the microchip 106, for example, to determine one or more of: a lifetime state of the inserted device (e.g., new, used, expired, exact age from manufacture, etc.); the type of the inserted device; the type of test sample expected on the inserted device; the analytes targeted for identification in the test sample; parameters for use in calculating a concentration of each targeted analyte. Upon receiving the identifying information from the microchip 106, the signal analyzer 200 may load all necessary data (e.g., corresponding standard curve parameters, algorithms, etc.) from a local memory storage of the computing device for use in interpreting an intensity of emission light detected from the fluorescent dyes and for calculating corresponding concentrations of the targeted analyte(s).

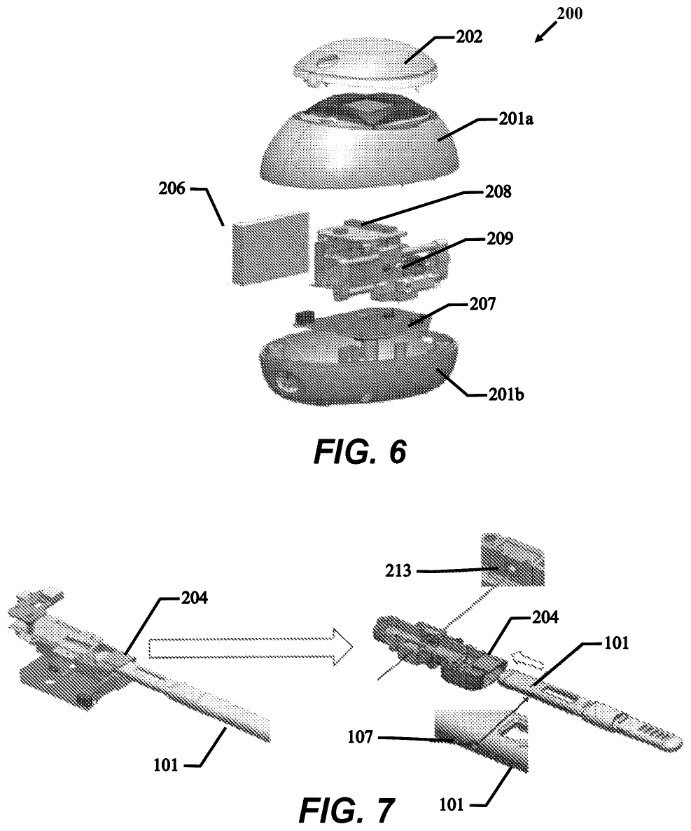

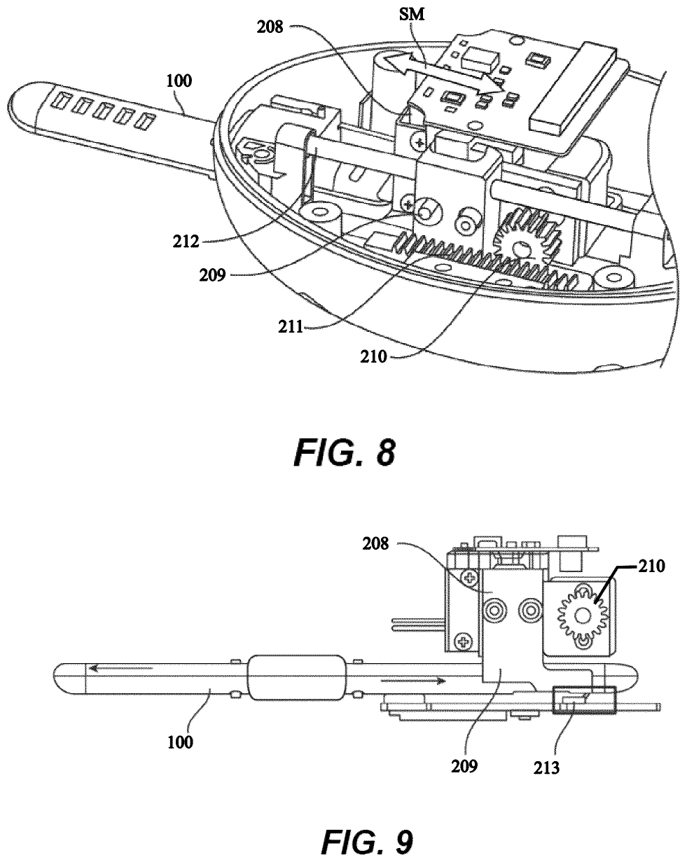

The signal analyzer 200, as seen in FIGS. 6-9, has a housing with a top panel 201a and a bottom panel 201b that enclose a local power source 206 (e.g., a battery), a computing device 207, an optical-mechanical detector module (OMDM) 208, and a motor 209 that drives a pinion 210 for translating the OMDM 208 in a scanning movement (SM) on a linear rack 211 and along linear guide rods 212. The computing device 207 includes a PCB assembly with microcontroller chips for executing programs and other processes and running firmware generally, a set of temperature and humidity sensors, a battery management module, a computational module, memory storage, and other electronic control devices for operation of the analyzer.

The OMDM 208 is configured such that, when an immunoassay device 100 is inserted into the insertion slot 204 of the signal analyzer 200, the OMDM 208 can transmit light (excitation and emission light) through the reading window 104 in the casing 101 of the immunoassay device 100 proximate the test zones 154 of the internal test strip 150. In this way, the OMDM 208 can deliver an excitation light to the fluorescent labels at test zones 154 and receive an emission light from the fluorescent dyes in those labels.

FIG. 7 shows an isolated view of insertion slot 204 of the signal analyzer 200 and an immunoassay device 100. The casing 101 of immunoassay device 100 is provided with a cavity 107 and an internal surface of the insertion slot 204 is provided with a spring-loaded a ball bearing 203. Upon insertion of immunoassay device 100 into insertion slot 204, the ball bearing 203 may insert into cavity 107, thereby locking the immunoassay device 100 into insertion slot 204. The signal analyzer 200 may include a positioning switch 213 configured to switch between an open position and a closed position. The positioning switch 213 may be in an open position when the immunoassay device 100 is not inserted into the insertion slot 204 and may be in a closed position when the immunoassay device 100 is inserted a predetermined depth into the insertion slot 204. The predetermined depth may correspond with a position at which the ball bearing 203 within insertion slot 204 engages cavity 107 on the immunoassay device 100. The positioning switch 213 may be configured to activate the motor 209 to drive the OMDM 208 to scan the test strip 150 of the inserted immunoassay device 100 when the positioning switch 213 is in the closed position. The scan of test strip 150 may be terminated if the scan has been completed, or when the positioning switch 213 has been moved to the open position by withdrawal of the immunoassay device 100 from insertion slot 204.

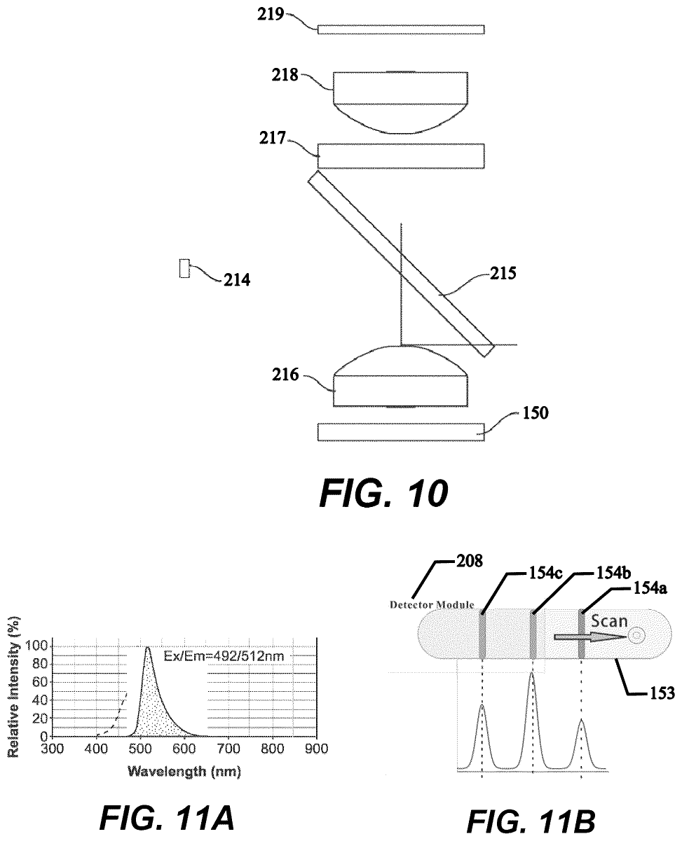

As seen in FIG. 10, OMDM 208 includes one or more light sources 214 (e.g., light emitting diodes, LED) for generating an excitation light. The excitation light emitted from the light source 214 is redirected by a dichroic mirror 215 to pass through a first lens 216 and a reading window 104 in the casing 101 of an immunoassay device 100, for delivery to the test zones 154 of the internal test strip 150, thereby exciting fluorescent dyes in fluorescent labels attached to the targeted analytes that have been captured at the test zones 154. The excited fluorescent dyes generate an emission light that passes through the reading window 104 in the casing 101 of the immunoassay device 100 and travel though the first lens 216 toward the dichroic mirror 215. The dichroic mirror 215 is provided with a series of optical filters that permit only the emission light (or a fraction thereof) to pass therethrough, with the emission light then passing through a filter 217 and a second lens 218 for reception at a photodetector 219. Photodetector 219 communicates with an electronic module to convert the received emission light into an output signal representative of fluorescence intensity that is then directed to computing device 207.

The fluorescent intensity of the emission light received at the photodetector 219 may vary depending on the type, dispersion, and concentration of the fluorescent dye within the test zones 154. The motor 209 drives the pinion 210 for translating the OMDM 208 back-and-froth on the rack 211, along an optical axis corresponding with the linear guide rods 212, to perform multiple scanning movements of the OMDM 208 over the test zones 154 exposed through the reading window 104 of the inserted immunoassay device 100. The OMDM 208 may capture varying fluorescence intensities at multiple locations along each single scanning movement over the test zones 154. With capture of these varying intensities, the computing device 207 may generate one or multiple signal detection curves. FIG. 11A shows a signal detection curve plotting a relative signal intensity versus signal wavelength, and FIG. 11B shows a signal intensity curve with multiple signal intensity peaks, corresponding with multiple test zones 154a, 154b, 154c of an immunoassay device 100, plotting fluorescence intensity versus position. The computing device 207 may use the signal processing program stored at the local memory to process signal curve data to calculate peak heights, areas, sizes and baselines based on the predetermined parameters that were identified from the information stored at the microchip 106 on the immunoassay device 100 and retrieved from the local memory of the computing device 207. The signal processing program may calculate concentration levels of the targeted analytes from the test sample that was delivered to immunoassay device 100. The signal analyzer 200 may then display the test results on the display screen 202 and may further store and sync those results with prior-stored historical test results—this may be done locally at one or more personal devices and/or at one or more remote devices.

The computing device 207 may be further programmed to assess flow characteristics of a test sample through a test strip 150 of an immunoassay device 100 to ensure the test sample has properly distributed across the test zones 154 before generating immunoassay test results. Upon introducing a test sample to the sampling pad 151 of the test strip 150 through the sample collection region 103 of the immunoassay device casing 101, the test sample will begin migrating along the test strip 150 via capillary action promoted by the absorbent pad 155. Following introduction of the test sample to the sampling pad 151, the immunoassay device 100 is inserted in the insertion slot 204 of the signal analyzer 200 at which time information on the microchip 106 of the immunoassay device 100 is read by the computing device 207, via the calibration module. Computing device 207 uses data read from microchip 106 to identify and load necessary data from the local memory storage for performing an immunoassay analysis corresponding to the specific inserted immunoassay device 100.

After a first time period T1 (e.g., 0 seconds, 1 second, 5 seconds, etc.), OMDM 208 is driven along the linear guide rods 212 and over the reading window 104 of the inserted immunoassay device 100 to perform a first optical scan of the test zones 154 on the test strip 150 stored within the immunoassay device 100. During the optical scan, the OMDM 208 delivers an excitation light to excite fluorescent dyes at test zones 154, triggering the exited fluorescent dyes to emit an emission light that is then detected by the OMDM 208. The OMDM 208 generates an output signal corresponding with the detected emission light and transmits the output signal to the computing device 207, which then uses a computational module to generate a signal curve corresponding with the intensity of the detected emission light (e.g., a fluorescent signal intensity curve). The OMDM 208 performs repeat optical scans at subsequent time periods (e.g., T2, T3, . . . . Tn), which may be at any chosen interval of time, such as 30 seconds, 1 minute, 5 minutes, 15 minutes, etc., with a signal curve generated for each optical scan. The time periods for subsequent optical scans need not be taken at equal intervals of time and may be adjusted depending on determinations made by the computing device 207. For example, a second optical scan may be performed at a time T2=1 minute, and a third optical scan may be taken at a time T3=5 minutes.

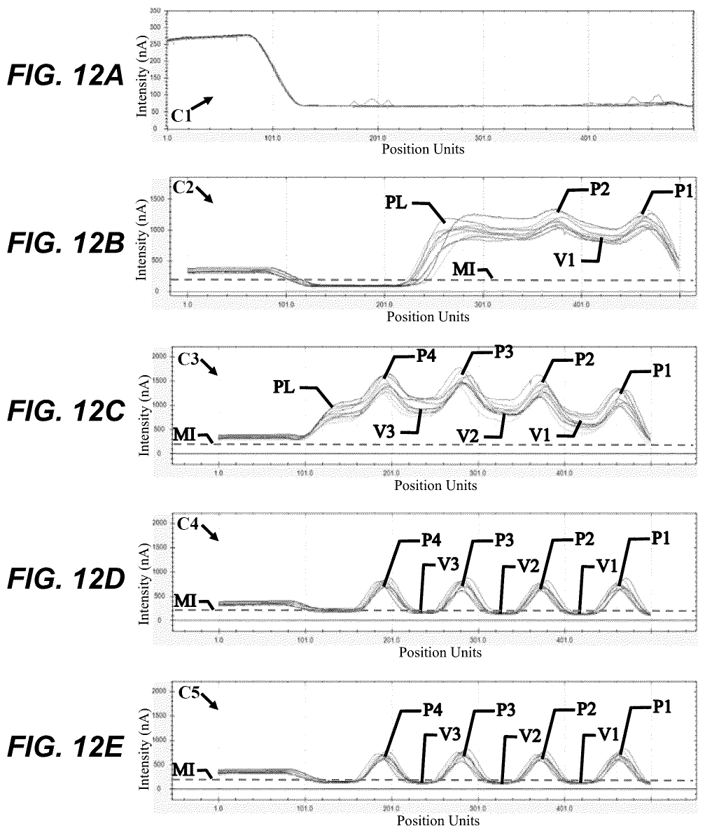

FIGS. 12A-12E show an example of signal curves C1-C5 generated from repeat optical scans performed by an OMDM 208 on a single immunoassay device 100. The optical scans plot fluorescent intensity (nA) versus predetermined positions (unit) along the inserted immunoassay device 100. The predetermined positional units may be any chosen unit of position measurement that is determined in advance for use with the system. In the curves C1-C5, the region from approximately 1.0 unit to approximately 80.0 units corresponds with an outer surface of the casing 101 of the immunoassay device 100; the region from approximately 80.0 units to approximately 120.0 units corresponds with a sloped surface between the outer surface of the casing 101 and the opening of the reading window 104 of the immunoassay device 100; and the region from approximately 120.0 units to approximately 500.0 units corresponds with a region of the nitrocellulose membrane 153 where there is located four separate test zones 154 (154a, 154b, 154c, and 154d), and which is exposed through the reading window 104. Curve C1 shows results from a first scan performed at a time T1=0 seconds; curve C2 shows results from a second scan performed at a time T2=1 minute; curve C3 shows results from a third scan performed at a time T3=5 minutes; curve C4 shows results from a fourth scan performed at a time T4=10 minutes; and curve C5 shows results from a fifth scan performed at a time T5=15 minutes.

As seen in FIG. 12A, the curve C1 generated at time T1=0 seconds is substantially flat, without any discernable peaks, and with a substantially uniform intensity of approximately 70 nA in the region from approximately 120.0 units to approximately 500.0 units. This flat profile and uniform intensity of curve C1 is due to the test sample with the fluorescent labels having not yet reached the test zones 154 of the test strip 150.

As seen in FIG. 12B, curve C2 generated at time T2=1 minute has elevated intensities in the region from approximately 220.0 units to approximately 500.0 units, with a plateau PL having an intensity of approximately 1200 nA at approximately 260 units and two peaks P1 and P2 both having an intensity of approximately 1300 nA at approximately 460 units and 380 units, respectively. As seen in FIG. 12C, curve C3 generated at time T3=5 minutes has elevated intensities in the region from approximately 101.0 units to approximately 500.0 units, with a plateau PL having an intensity of approximately 1000 nA at approximately 130 units and four peaks P1, P2, P3, and P4 having respective intensities of approximately 1200 nA, 1500 nA, 1600 nA, and 1500 nA at approximately 470 units, 370 units, 280 units, and 190 units, respectively.

As seen in FIG. 12D, curve C4 generated at time T4=10 minutes has elevated intensities in the region from approximately 160.0 units to approximately 500.0 units, with no apparent plateau ahead of four peaks P1, P2, P3, and P4 each having an intensity of approximately 700 nA at approximately 470 units, 370 units, 280 units, and 190 units.

As seen in FIG. 12D, curve C5 generated at time T5=15 minutes is substantially similar in appearance to curve C4, with elevated intensities in the region from approximately 160.0 units to approximately 500.0 units, with no apparent plateau appearing ahead of four peaks P1, P2, P3, and P4 each having an intensity of approximately 700 nA at approximately 470 units, 370 units, 280 units, and 190 units.

Though the curves C1-C5 are shown to eventually normalize with each of the peaks P1, P2, P3, and P4 having a common intensity value, it will be understood that the individual peaks may have different intensity values, for example, due to differences in the type of fluorescent labels associated with each test zone and the concentration of fluorescent labels captured at each test zone.

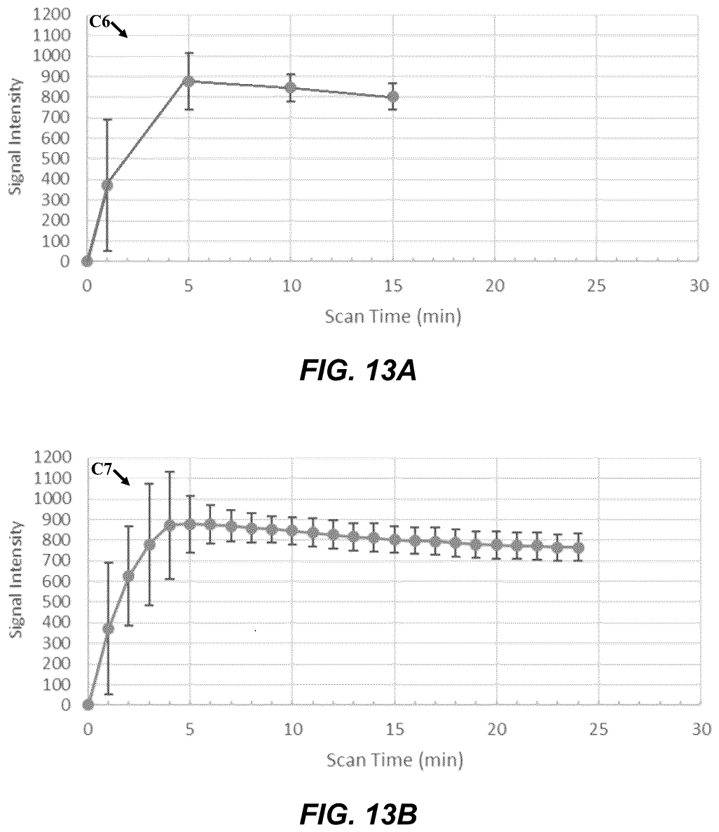

The data downloaded from the local memory storage by the computing device 207, based on data read from the microchip 106 on the immunoassay device 100, may include a count of the test zones 154 on the immunoassay device 100 as well as positioning data for each test zone 154. In the example of the immunoassay device 100 that resulted in curves C1-C5 above, the data downloaded by the computing device 207 may include information indicating there are four test zones 154 located at approximately 470 units, 370 units, 280 units, and 190 units. With the data identifying the number and location of each test zone 154, computing device 207 may generate one or more test-zone-specific (TZS) signal curves plotting signal intensity at a specific test zone versus scan time. For example, based on the signal curves C1-C5, a TZS signal curve C6, such as that shown in FIG. 13A, could be created for one of the four test zones 154 showing signal intensities measured at that specific test zone for each of the scan times T1-T5.

It is noted that FIGS. 12A-12E and 13A illustrate an example in which only five optical scans are performed at five time periods T1-T5, resulting in the TZS signal curve C6 having only five data points. These examples used only five optical scans for simplifying the above discussion, and it is expected, though not required, that systems according to the present invention will more regularly use more than five optical scans. For example, the system may perform an optical scan every minute for at least ten minutes, for at least fifteen minutes, for at least twenty minutes, etc. FIG. 13B shows an example of an TZS signal curve C7 that is generated from twenty-four separate optical scans performed once every minute.

With a TZS signal curve, computing device 207 may monitor changes in signal intensity at a test zone as a function of time to thereby assess flow of the test sample carrying the fluorescent label across the respective test zone. For example, based on curve C7 in FIG. 13B, computing device 207 can determine that: [a] a quick increase in intensity in the time range from zero to approximately four minutes corresponds with increasing concentration of the corresponding target analyte at the target zone; [b] a gradual decrease in intensity in the time range from approximately six minutes to approximately nineteen minutes corresponds with passage of labeled analytes without capture at the target zone; and [c] a substantially constant intensity after approximately nineteen minutes represents a final concentration of the labeled analyte captured at the target zone.

When generating the TZS signal curve 7, computing device 207 will compare successive intensity values in the TZS curve to assess a change in intensity over time. If the computing device 207 determines that a change in intensity between successive scans is above an intensity differential threshold value IA, then the computing device 207 may instruct the OMDM 208 to continue performing successive scans until such time that a change in intensity between successive scans falls below the intensity differential threshold value IA. The computing device 207 may also instruct the OMDM 208 to alter one of more parameters of the further optical scans. For example, the computing device 207 may instruct the OMDM 208 to change a time interval between successive scans to either increase or decrease the time between scans depending on a detected rate of change in the signal intensity. Optionally, once the computing device 207 determines that a change in intensity between successive scans falls below the intensity differential threshold value IA, the computing device 207 may instruct the OMDM 208 to perform a predetermined number of additional scans (e.g., one, three, five, ten additional scans, etc.) to obtain additional data points for ensuring the change in intensity between successive scans remains below the intensity differential threshold value IA. Optionally, while continuing to perform optical scans, the computing device 207 will output a message to the display screen 202 instructing that the immunoassay device 100 be left inserted into the insertion slot 204, and once optical scanning is complete the computing unit 207 may output a message to the display screen 202 instructing removal of immunoassay device 100 and/or displaying the test results.

The PCB computing device 207 may use a TZS curve 7 to confirm proper flow of the test sample through the immunoassay device 100 and ensure test results are calculated under a proper, error-free operation of the system, with sufficient time for the test sample to flow across all test zones 154. For example, if there is no increase in intensity at one or more test zones 154, then computing device 207 may determine that the test sample did not properly reach the one or more corresponding test zones 154. If there is an increase in intensity followed by a rapid decrease in intensity, or a failure in achieving a substantially constant intensity after a predetermined maximum time period (e.g., T=30 minutes), then computing device 207 may determine there has been a failure of the capture reagents to properly capture the targeted analyte at the test zone. When an error is identified, the computing device 207 may output a message the display screen 202 indicating an error in the test and/or instructing that a new test be performed with a new immunoassay device 100.

Computing device 207 may generate a TZS signal curve 7 for each test zone 154 in an immunoassay device 100 to thereby monitor for errors relative to each of the test zones 154 and ensure accurate test results for each of the corresponding targeted analytes. When using multiple TZS signal curves 7, computing device 207 may instruct the OMDM 208 to perform additional optical scans until an intensity change between successive scans in each TZS curve 7 is determined to fall below a corresponding predetermined intensity differential threshold value IA.

Optionally, in addition to assessing peak intensity changes via TZS curves 7, computing device 207 may also measure intensity changes at valleys of the signal curves C1-C5. Regions of the nitrocellulose membrane 153 between the individual test zones 154 will be free of capture reagents, resulting in minimal to substantially no retention of fluorescent labelled analytes in those intermediate regions after passage of the test sample. While the test sample continues to flow through test strip 150, optical scans by the OMDM 208 will show elevated signal intensities in the intermediate regions, which will appear as valleys between signal peaks. Sec, for example, valleys V1, V2, and V3 in curves C2 and C3 of FIGS. 12B-12C. After flow of the test sample through test strip 150 is completed, optical scans by the OMDM 208 will show minimal signal intensities in those intermediate regions, which will appear as valleys of substantially uniform intensity between signal peaks. See, for example, valleys V1, V2, and V3 in curves C4 and C5 of FIGS. 12D-12E. The computing device 207 may monitor signal intensities in these valleys, similar to monitoring of signal intensities at the adjacent peaks, though without need to assess changes in intensity at the valleys between successive scans and instead assessing only whether intensities at the separate valleys drop to levels below a minimum intensity threshold level MI. Computing device 207 may identify a drop in intensity at the valleys to a level below the minimum intensity threshold level MI as indicative that there is little to no fluorescent label remaining in those intermediate regions, as the test sample is no longer flowing through the test strip 150, thereby informing that the signal intensities at the peaks should therefore be indicative of a final concentration of captured analytes at the test zones 154.

Advantageously, a single signal analyzer 200 may locally store data (e.g., corresponding standard curve parameters, algorithms, etc.) and algorithms for performing multiple different types of analyte tests and may load the necessary data and algorithms for specific analyte tests based on information retrieved from the microchip 106 on an immunoassay device 100 that is inserted into the insertion port 204 of the signal analyzer 200. In this way, a single signal analyzer 200 may be adapted to work with multiple different types of immunoassay devices 100 for performing multiple different analyte tests and may be adapted to concurrently test a single test sample for multiple separate analytes. A signal analyzer 200 may store the necessary data and algorithms for concurrently performing multiple separate analyte tests based on signals from a single immunoassay device 100 to concurrently test a single test sample (e.g., a urine sample) for four separate analytes (e.g., LH, E3G, FSH, and PdG).

The signal processing program may include programming stored at one or more of: locally at the signal analyzer 200 (e.g., firmware); personal user devices (e.g., mobile application software); third-party service provider devices (e.g., application software on healthcare provider devices); and one or more remote central service systems (e.g., backend software).

In a preferred example, the signal processing program includes programming at the signal analyzer 200 that communicates with programming at one or more personal user devices via either wired or wireless communication protocols. Programming at a personal user device communicates with programming at the central service system, and optionally programming at third-party service provider devices, preferably through wireless communication protocols. Suitable wireless communication protocols include, though are not limited to: Bluetooth, Wi-Fi, cellular signals, internet protocols (e.g., HTTPS), etc.

Once the signal analyzer 200 has assessed and generated results from the signal outputs of an immunoassay device 100, the testing results and/or error information and/or warning information generated therefrom (collectively, result data) is stored in a memory of the signal analyzer 200. The signal analyzer 200 may then transmit the result data to a user's personal device, which may then store and sync the result data with the programming stored locally thereon (e.g., firmware). Programming at the user's personal device may then transmit the result data to one or more central service systems (e.g., service provider servers), and optionally to one or more third-party service provider devices (e.g., healthcare provider portal). Optionally, the transmission of result data may be conditioned in each instance on approval or instructions from the user, for example, with approval for transmitting from the signal analyzer to a mobile phone, and further approval before transmitting from the mobile phone to either the service provider or the healthcare provider.

Upon receiving result data, programing at the central service system processes concentrations of the targeted analytes. The programing at the central service system may further sync the resulting concentration values with other complimenting data to provide additional information. For example, when the targeted analytes are hormones that are informative of conception, then the programing at the central service system may sync the concentrations of those hormones with menstrual cycle information and may then provide further assessments based on the combined data. For example, the combined data may be used to provide fertility assessments, contraception guidelines, and ovulation predictions; identify potential health conditions like polycystic ovary syndrome (PCOS) or hormone imbalances; and/or aid in tracking treatment progress. The combined data may further be used to provide statistical analysis of hormone trends, comparisons against established population groups, and diagnoses of medical conditions such as: PCOS; menopause; adrenal disorders, such as congenital adrenal hyperplasia, ovarian or adrenal tumors, congenital adrenal hyperplasia (CAH), as informed by elevated 17-OH progesterone; and other hormone imbalances.

The programing at the central service system may also employ a dynamic threshold system, as a substitute for a standard (fixed) threshold system. In a standard threshold system, the programming would establish a threshold reading based on a wide population of common test subjects. This may be helpful in some instances, as it enables the use of data from a larger pool of test subjects, which would theoretically provide more accurate testing for the population at large. However, such standard threshold systems are poorly suited for use with outlier test subjects that have conditions not normally found in the larger population.

For example, when the system and programming is adapted for use in tracking hormones related to conception, a standard threshold system may be undesirable as there can be significant variability in urinary hormone levels from one subject to the next, and even between individual menstrual cycles in a common subject. A dynamic threshold system may be better suited for such circumstances, as it can adapt to a baseline status for the tested subject to thereby enable assessments that are more precise to that specific subject. In such use scenarios, the programing may process menstrual cycle information of the specific subject, such as start and end days of the menstrual cycle, along with the length of both the menstrual cycle and the period, and then combine that data with the hormone concentration data for a more comprehensive analysis. By combining this subject-specific data, the program may provide custom-tailored feedback to a user, including a fertility scoring, menstrual phase detections, and even detailed health-related observations and guidance tailored to the specific user. The program may further aid in monitoring treatment progress and outcomes, support artificial reproductive technologies such as in vitro fertilization (IVF) and intrauterine insemination (IUI), schedule further conception testing (via the immunoassay device), and deliver additional insights related to early and high-risk pregnancy, including predication of miscarriage. By focusing on subject-specific data and patterns and integrating multi-dimensional data using preparatory machine learning models and artificial intelligence algorithms, the program may ensure more precise and individualized assessments over those that would otherwise be available from systems that employ standard threshold systems that rely on fixed data points derived from a larger population of test subjects.

Third-party service providers (e.g., healthcare providers) may be provided with dedicated devices/systems for communicating directly with personal user devices or may instead be provided with remote access portal to specific user data. A remote access portal may provide access to data stored at the central service system, and fetch and display the result data and processed data. A service provider may use a remote access portal only to access data from the central service system for users that have given advance authorization for such access (e.g., users that are acknowledged patients to the specific healthcare provider). Alternatively, a remote access portal may provide direct communication between a third-party service provider and a user's personal device.

The present invention is inclusive of, and may be characterized by, separately or in any combination of, the following aspects, either in whole or in part.
(1) A signal analyzer and a number of immunoassay devices form a system that measures the quantitative concentration of LH, E3G. FSH and PdG hormone levels simultaneously, by testing with one immunoassay device. Wherein each single immunoassay device is loaded with anti-LH, anti-E3G, anti-FSH and anti-PdG antibodies, E3G-protein conjugated complexes, and PdG-protein conjugated complexes. Wherein the signal analyzer is adapted to read the specific immunoassay devices, including information stored in the embedded microchip and fluorescent signals from the test zones of the immunoassay devices. The signal analyzer being further adapted to output quantitative concentrations of LH. E3G. FSH and PdG hormone levels. The output results may be displayed on the display screen of the signal analyzer and/or synced with or stored by the application software.
(2) The application software takes the LH, E3G, FSH, and PdG quantitative hormone levels simultaneously, or a subset of these four hormones (one hormone or two-hormone combinations) as inputs and adapts dynamic thresholds for each hormone. The dynamic thresholds are subject-specific, varying from user to user. The dynamic thresholds may vary over the course of use for a single user to adapt to hormone changes for the specific user over a specific time range. Wherein the dynamic thresholds combined with the LH, E3G, FSH, and PdG hormone levels are used to calculate fertility information, including one or more of: [i] a fertility score, which indicates the fertility level (e.g., ranging from 1-10; with one number assigned to each day in a cycle, with “1” for a least fertile day and “10” for a most fertile day); [ii] fertile vs. non-fertile days; [iii] prediction of an ovulation day, period days, and fertility level of each day, for the current and future cycles; [iv] detection of the period phase, follicular phase, and luteal phase; [v] detection of fertile window; [vi] confirmation ovulation has happened; and [vii] detection of when an ovulation happens.
(3) The application software takes the LH, E3G, FSH, and PdG quantitative hormone levels simultaneously, or a subset of these four hormones (one hormone or two-hormone combinations) as inputs, and outputs fertility information of the user. Wherein the application software takes into account menstrual cycle information, including but not limited to the start and the end days of the cycle, the start and end days of the period days, the length of the cycle and the period, combining this information with the measured hormone levels in Aspect 1, and outputs fertility information, including one or more of: [i] a fertility score, which indicates the fertility level (e.g., ranging from 1-10; with one number assigned to each day in a cycle, with “1” for a least fertile day and “10” for a most fertile day): [ii] fertile vs. non-fertile days: [iii] prediction of an ovulation day, period days, and fertility level of each day, for the current and future cycles; [iv] detection of the period phase, follicular phase, and luteal phase; [v] detection of fertile window; [vi] confirmation that ovulation has happened; and [vii] detection of when an ovulation happens. Wherein the application software takes into account the menstrual cycle information, including but not limited to the start and the end days of the cycle, the start and end days of the period days, the length of the cycle and the period, and personal information, including but not limited to the user's age, pathological conditions, physiological conditions, and lifestyle information, combining this information with the measured hormone levels in Aspect 1, and outputs the personalized information, including one or more of: [i] health-related educational content, including but not limited to health tips, articles, images, videos, courses, conversations etc.; [ii] health-related action guide and plan (meditation plan, nutritional plan, exercise plan, by weeks in a cycle, for example); and [iii] initiation of health-related chat, consultation, coaching, and telemedicine.
(4) Statistics analyses of the LH, E3G, FSH, and PdG hormones, or a subset of these four hormones (one or two or three-hormone combinations). Wherein the output information includes one or more of: [i] the trend of change of hormones over time, presented as data, table, or graph; [ii] statistics and calculations of the population average, variance, standard deviation, and outliers of the hormone levels; [iii] detections and analyses of the healthy and pathological hormone profiles; [iv] comparison of the user's tested hormone levels with the hormone profile of the corresponding population groups, including but not limited to healthy, trying to conceive, pregnant, post-miscarriage, postpartum, breastfeeding, perimenopause, menopause, post-menopause, PCOS, hormone imbalances, irregular cycles, and under any fertility treatments or hormonal treatments; and [v] generation, presentation, and sharing of the reports based on information above.
(5) The application software takes the LH, E3G, FSH, and PdG quantitative hormone levels simultaneously, or a subset of these four hormones (one hormone or two or three-hormone combinations), cycle information—including but not limited to one or more of: [i] the start and the end days of the cycle; [ii] the start and end days of the period days; and [iii] the length of the cycle and the period—in combination with the measured hormone levels in Aspect 1, as inputs, and outputs contraception information. Wherein the contraception information includes one or more of: [i] a fertility score, which indicates the fertility level (e.g., ranging from 1-10; with one number assigned to each day in a cycle, with “1” for a least fertile day and “10” for a most fertile day); [ii] fertile vs. non-fertile days; [iii] prediction of an ovulation day, period days, and fertility level of each day, for the current and future cycles; [iv] detection of the period phase, follicular phase, and luteal phase; [v] detection of fertile window: [vi] confirmation that ovulation has happened; and [vii] detection of when an ovulation happens.
(6) The application software takes the LH, E3G, FSH, and PdG quantitative hormone levels simultaneously, or a subset of these four hormones (one hormone or two or three-hormone combinations), menstrual cycle information-including but not limited to the start and the end days of the cycle, the start and end days of the period days, the length of the cycle and the period-in combination with the measured hormone levels in Aspect 1, as inputs, and outputs a medical diagnosis. Wherein the medical diagnosis includes one or more of: [i] polycystic ovary syndrome (PCOS): [ii] miscarriage; [iii] recovery of the postpartum; [iv] perimenopause, menopause, post-menopause; [v] hormone imbalances or irregular cycles; and [vi] infertility.
(7) The application software takes the LH, E3G, FSH, and PdG quantitative hormone levels simultaneously, or a subset of these four hormones (one hormone or two or three-hormone combinations), cycle information-including but not limited to the start and the end days of the cycle, the start and end days of the period days, the length of the cycle and the period in combination with the measured hormone levels in Aspect 1, as inputs, and outputs tracking of the treatment progress and outcome predictions. Wherein treatment progress and outcome include treatments and outcomes of treatments for one or more of: [i] polycystic ovary syndrome (PCOS); [ii] miscarriage; [iii] recovery of the postpartum; [iv] perimenopause, menopause, post-menopause; [v] hormone imbalances or irregular cycles; and [vi] infertility.

(8) The application software takes the LH, E3G, FSH, and PdG quantitative hormone levels simultaneously, or a subset of these four hormones (one hormone or two or three-hormone combinations), cycle information-including but not limited to the start and the end days of the cycle, the start and end days of the period days, the length of the cycle and the period—in combination with the measured hormone levels in Aspect 1, as inputs, and outputs hormone levels and treatment progress and outcome to be used in the artificial reproductive technology (ART). Wherein treatment progress and outcome include one or more of: [i] intrauterine insemination (IUI); [ii] in vitro fertilization (IVF): [iii] egg freezing: [iv] frozen embryo transfer; and [v] detection of hyperstimulation.
(9) The application software takes the LH, E3G, FSH, and PdG quantitative hormone levels simultaneously or a subset of these four hormones (one hormone or two or three-hormone combinations) combined with the cycle information, including but not limited to the start and the end days of the cycle, the start and end days of the period days, the length of the cycle and the period, as inputs, and outputs a recommended immunoassay device testing schedule.
(10) The application software takes the LH, E3G, FSH, and PdG quantitative hormone levels simultaneously, or a subset of these four hormones (one hormone or two or three-hormone combinations) as inputs, and outputs pregnancy-related information. Wherein pregnancy-related information includes one or more of: [i] early pregnancy detection; [ii] miscarriage detection and prediction; [iii] high risk pregnancy detection and prediction; and [iv] ectopic pregnancy detection and prediction.

Although the present invention is described with reference to particular embodiments, it will be understood to those skilled in the art that the foregoing disclosure addresses exemplary embodiments only; that the scope of the invention is not limited to the disclosed embodiments; and that the scope of the invention may encompass any combination of the disclosed embodiments, in whole or in part, as well as additional embodiments embracing various changes and modifications relative to the examples disclosed herein without departing from the scope of the invention as defined in the appended claims and equivalents thereto.

To the extent necessary to understand or complete the disclosure of the present invention, all publications, patents, and patent applications mentioned herein are expressly incorporated by reference herein to the same extent as though each were individually so incorporated.

The present invention is not limited to the exemplary embodiments illustrated herein, but is instead characterized by the appended claims, which in no way limit the scope of the disclosure.

What is claimed is:

1. A lateral flow immunoassay system comprising:
an immunoassay device having a test strip for reception and migration of a test sample therealong, the test strip comprising: a sampling pad for reception of a test sample, conjugate pad carrying optical labels conjugated to antibodies specific to one or more target analytes in the test sample, and one or more test zones for capture of one or more target analytes;
a signal analyzer having a scanning mechanism configured to perform multiple optical scans of the one or more test zones of the test strip to detect optical signals from optical labels on one or more captured target analytes;
a computational module configured to generate signal detection curves based on optical signals detected from optical scans of the one or more test zones, compare signal detection curves to assess flow characteristics of the test sample along the test strip, and determine the signal intensities and concentrations of the target analyses.

2. The system of claim 1, wherein the optical label is a fluorescent label and the scanning mechanism is a fluorescence scanner.

3. The system of claim 1, wherein the computational module is configured to generate signal detection curves plotting optical signal intensity against test strip length for each optical scan.

4. The system of claim 1, wherein the computational module is configured to compare signal detection curves to identify variations in flow dynamics and analyte distribution of the test sample.

5. The system of claim 1, wherein the computational module is configured to adjust assay parameters based on the flow characteristics of the test sample.

6. The system of claim 1, wherein the computational module is configured to adjust assay parameters in real-time.

7. The system of claim 6, wherein the computational module is configured to adjust assay parameters that include the performance of additional optical scans of the one or more test zones.

8. The system of claim 7, wherein the computational module is configured to adjust assay parameters that include changing an interval of time between the additional optical scans.

9. The system of claim 1, wherein the computational module is further configured to detect occurrence of an error in an immunoassay test based on flow characteristics of the test sample.

10. A method of performing a lateral flow immunoassay, comprising:
introducing a test sample onto a sampling pad of a test strip;
allowing the test sample to migrate along the test strip via capillary action, with the test sample migrating through a conjugate pad of the test strip for labelling of one or more target analytes with an optical label and subsequently through one or more test zones of the test strip for capture of one or more target analytes;
performing multiple optical scans of the one or more test zones to detect emission signals from optical labels attached to the one or more target analytes captured at the one or more test zones, with individual optical scans performed at different time periods;
generating signal detection curves based on the emission signals detected from the optical labels in the multiple optical scans; and
comparing signal detection curves to assess flow characteristics of the test sample along the test strip and determining the signal intensities and concentrations of the target analyses.

11. The method of claim 10, wherein the optical label is a fluorescent label and optical scanning is performed with a fluorescence scanner.

12. The method of claim 10, wherein the signal detection curves plot optical label intensity against test strip length for each optical scan.

13. The method of claim 10, further comprising adjusting assay parameters based on the flow characteristics in real-time.

14. The method of claim 13, wherein adjusting assay parameters comprises performing additional optical scans of the one or more test zones.

15. The method of claim 14, wherein adjusting assay parameters further comprises changing an interval of time between the additional optical scans.

16. The method of claim 10, further comprising detecting occurrence of an error in an immunoassay test based on flow characteristics of the test sample.
